(19) United States
(12) Patent Application Publication
(10) Pub. No.: US 2008/0027752 A1
(43) Pub. Date: Jan. 31, 2008

(54) Physician reviewed portable and network accessed electronic medical record

(76) Inventors: Giang Trieu Phan, Queen Creek, AZ (US); William Crane, Phoenix, AZ (US); Peter Nguyen, Phoenix, AZ (US)

Correspondence Address: MICHAEL RIES, 318 PARKER PLACE, OSWEGO, IL, 60543, US

(21) Appl. No.: 11/496,203

(22) Filed: Jul. 31, 2006

(51) Int. Cl. G06Q 10/00; A61B 5/00

(52) U.S. Cl. 705/2; 600/300

(57) ABSTRACT

A system for managing, storing, and viewing medical records wherein the method includes a portable digital memory device, a network, a medical record database, and a physician review portal. The medical records are reviewed by a physician and summarized to a condensed form of essential elements of a medical record through remote computer connected to a network. The reviewed medical record is then transferred to a portable digital memory device that is carried by the patient. The reviewed medical record can be viewed through a remote computer connected to the network or through the portable digital memory device. Also, a software program on the server can extract core elements from an electronic medical record prior to physician review.

### FIELD OF THE INVENTION

The present invention relates to the field of managing electronic medical records through portable secure digital memory device and network accessed medical records, and more particularly, managing electronic medical records.

### BACKGROUND OF THE INVENTION

The importance of a portable or easily accessible medical record is important in emergency situations where patients are altered or unconscious. A treating emergency department physician or a medical field specialist (such as Cardiology) needs key medical information for effective management, avoiding medication errors, determining advanced directives for end of life wishes, prevent repeating laboratory tests that have been recently ordered. Additionally, outpatient office visit to medical specialists would minimize rescheduling patients due to waiting for transferring medical records that have not yet arrived. Thus, there exists a need for portable (carried by the patient) or easily accessible electronic medical record system.

A credible medical record cannot allow patients to alter important medical information such as medications or past medical history. For example, a narcotic abusing patient (if given the means) can alter their medical records to read that they have terminal cancer thus causing narcotics to be much easier to obtain from a physician. Additionally, a malingering patient may alter his medical record to help him claim disability. Given the abuse potential of allowing patients to alter key pieces of medical information, the lack of expertise in the medical field by the public, and inability to recall medical information accurately by the public, most physicians do not accept medical records entered or altered by patients as credible.

Currently, the majority of medical records are in paper format—a collection of handwritten notes of visits, laboratory data, reports of imaging, operations, procedures, etc. Reading the medical chart of complicated patients can be difficult due to the volume of pages, an unorganized system of records, illegible handwriting, and a lack of summary pages of key medical information. Reviewing medical records can also be complicated. Although many people in the healthcare field can review medical charts, the person that can perform the most accurate review is probably physicians. Additionally, spending excessive time reviewing an unorganized medical record by emergency department physicians or the allotted office time of outpatient physicians can hinder and decrease the quality of healthcare. Thus, there exists a need to convert paper charts to electronic medical records and a need to create a portable or easily accessible summarized electronic medical record.

There is agreement among physicians regarding the elements of a summarized medical record. Many of these elements are found in the standard “history and physical” form used by most physicians. Governing bodies of medicine (American Academy of Family Physicians, American Academy of Pediatrics) have adopted standards for key elements that should be found in an abbreviated/summarized version of a medical record. An example includes CCR (Core Continuity Records, from American Society for Testing and Materials). These records allow standardization and the ability to transfer electronic health records between electronic medical records (EMRs) and portable medical records.

An effort to allow the easy transfer of electronic medical records from different electronic medical record keeping systems, the government has mandated a standardization of electronic medical records in terms of the method in which the data files are stored. Standards are being devised by the government and medical organizations. An example is HL7 (Health Level 7). In terms of a compact, summarized version of a complete medical record, it will be possible in the future to write one software program to extract the core elements of an electronic medical record.

Medical records must be Health Information Portability and Accountability Act compliant (HIPAA). HIPAA has strict policies on the transmission, the viewing, and requesting patient protected health information. In general, HIPAA encourages protecting medical information against unauthorized viewers, requires consent prior to the release of medical records, and has penalties for violating its laws.

The features of an ideal portable medical device containing an electronic medical record as follows. First, it must have a method to prevent the viewing of information by unauthorized users. For example, misplacing or losing the portable medical record device could enable confidential medical information to be viewed by another person if not secured. One security method is the requirement of a logon name and password to access the data files. Second, the data files on the portable medical record must be secured against alteration by unauthorized users. For example, pdf (portable document format) records can copied and altered with readily available software (Adobe Acrobat). One method to prevent the altering of medical record data files is encryption. Third, the portable medical record device must not install software on a remote computer of a hospital, doctor's office, or the emergency department. Many of these computers have firewalls or security protection that prevents unauthorized installation of software, thus making the software application not executable. Fourth, the portable medical record device has to be easily read by hardware in the majority of healthcare provider computers. One method is to utilize a jump drive which has a USB port. This port is very common in most U.S. computers. Card and microchip readers are not readily available at doctor's offices. In addition, CD ROM and disks are too large or too awkward to be transported by patients.

Internet displayed and transferred medical records must have security features that prevent access to unauthorized third parties. A method of identifying the patient and the verification of the identity of the requesting physician or doctor's office requesting the records must be in place.

It is therefore the object of the present invention to provide a system and method for providing personal and medical information that is credible, portable, easily accessible, HIPAA compliant, reviewed and summarized of continuity core elements, tamper proof by patients, and easy to update by authorized medical providers.

U.S. Published Pat. Application 20050108059 entitled “Portable Health Data System” which published on May 19, 2005 to Tay describes a health data system whereby personal health information is stored and transferred on a USB memory drive from a connection to a web database through an intermediate electronic system such as a personal computer or workstation. The encrypted personal health data files are entered by the patient. The method of viewing allows the data files to call common applications already stored on a remote computer that reads or displays that type of data file. There is no internal viewer application software on the USB drive.

U.S. Published Pat. Application 20060015368 entitled “Portable Method and Device for Personal Medical Record Compilation and Retrieval” which published on Jan. 19, 2006 to Hockey describes storing portable document file (PDF) files without password protection and unencrypted on a USB memory drive where an existing general application file on the remote computer views the stored data files. One drawback is that not all computers may have the required application for reading and viewing the PDF files. Another aspect is that patients can alter their medical record be changing or reloading doctored PDF files. The lack of username and password and encryption of the data file allows anyone to easily view the files if the USB memory device is lost. This violates HIPAA policy.

U.S. Published Pat. Application 20040103000 entitled “Portable System and Method for Health Information Storage, Retrieval, and Management” which published on May 27, 2004 to Owurowa et al. describes a method to transport medical records through a portable USB device and a method for encrypting the stored data on the USB device. Disadvantages of this invention are that it allows patients to enter their own medical records, it installs software on remote computers that may not allow it due to security and firewall protection, and it releases only a partial amount of medical information without releasing the name of the person on the USB device.

U.S. Published Pat. Application 20060080137 entitled “USB Med Stick with Personal Medical History” which published on Apr. 13, 2006 to Chambers at al. describes a USB device with a password protected software application that displays a person's medical history when inserted into a computer. A person fills out personal information and their own medical history from a website, which is stored on an internet database. The encrypted data file containing the medical information and the software application is downloaded to a memory stick belonging to the patient. In the event of an emergency, a red button on the welcome page allows access to “information necessary to save your life”. One drawback is that the medical information is patient entered, without official medical record documents, laboratory, records, or imaging reports from a physician's office.

U.S. Published Pat. Application 20030208382 entitled “Electronic Medical Record System and Method” which published on Nov. 6, 2003 to Westfall describes a system for a person to alter or update their own medical record, view their medical record anytime, and a method for synchronizing the records through a “portable data access device” (such as a personal digital assistant, palm top device, handheld computer, desktop computer, cellular phone) with an internet server database.

U.S. Published Pat. Application 20060010012 entitled “Portable Medical Information Device” which published on Jan. 12, 2006 to Franzblau et al. describes a system and a method for providing patient to enter their own medical and personal information on storage element, preferably a with USB port interface. The executable software on the storage element is not username password protected and the data files are not encrypted.

U.S. Published Pat. Application 20030088439 entitled “Portable Personal Health Information Package” which published on May 8, 2003 to Grushka describes a health information management system where the health information is organized and viewed by a portable computer storage device organized in the body system. The system allows patients to enter their own medical data. There is no mention of username or password, data file encryption or a web based database to store or draw the information.

U.S. Published Pat. Application 20060085347 entitled “Method and Apparatus for Managing Personal Medical Information in a Secure Manner” which published on Apr. 20, 2006 to Yiachos describes a method for access to privileged medical information classified in tiers on a portable secure media device. The device sends to a web server a digital security key after a username and password is entered. It also describes a method whereby more sensitive information (second tier information) is sent to a qualified physician after credentials are verified. One disadvantage is that the digital key verification, username and password verification of the portable secure media device through the internet with a web server, does not function if the computer of the treating physician is not connected to the internet.

### BRIEF SUMMARY

The present invention has been devised to provide a method for processing and managing personal and medical record information. Specifically, the present invention provides a computer management system that provides people to carry their latest medical and personal information on a portable digital memory device. It devises a method for reviewing and summarizing medical records to a smaller core record by physicians and placing the reviewed medical record on a portable digital memory device and access through a network such as the internet.

According to one aspect, paper medical records are faxed or e-mailed to a network server such as internet or intranet. The records are then queued and sent to a physician who reviews and summarizes the records. There is a physician portal that allows both viewing and data entry from a secured connection from a remote computer. The server then accepts the reviewed medical records and stores it in a medical record database. A person who subscribes to the service can elect to have their records on a portable digital memory device or accessed via a network (intranet or internet, but preferably the internet) to their reviewed medical records. The reviewed medical records stored on the medical record database can be downloaded or transferred to the portable digital memory device through the network to a remote computer.

The purpose of having portable medical records and network accessed medical records is to allow for higher quality of care, avoid medical errors, and give advanced directives in emergent situations. If the patient is conscious, they can give their username and password and the portable digital memory device to a physician or medical staff. If they are not carrying their portable digital memory device, they can ask the physician to access the medical records through the network such as the internet. If the patient is unconscious, directions to call a 1-800 number on a or go to a website for instructions on how to get emergent access will be engraved or written on a bracelet, necklace or a piece of jewelry. The person signs a release of medical records authorization when they sign up for the service. The physician's information is verified which can include DEA number, medical license number, address, and phone. Once the information is verified, a temporary username and password is given to access either the portable digital memory device or the network for a short period of time, such as 24 hours.

In the future, when electronic medical records must conform to a set standard (such as HL7), it will be possible to write one program to access the medical records and pull the essential information from the electronic medical records. One feature of the present invention is an extraction program that will preliminarily extract essential medical data from fields of the electronic medical record. It will then be subjected to physician review prior to storing the records on the medical records database.

The present invention gives the highest security possible. It does not allow people to change or input their own medical records, allowing this would invalidate the records. The medical record data files on the portable digital memory device are encrypted. They are “read only” type of files. There is a viewer application on the portable digital memory device that requires a username and password for access. The reviewed medical records are stored in encrypted format on the network database.

There are many objects the invention accomplishes. First, the present invention allows a portable device in which patients can carry their own medical records. Second, the present invention allows records to be condensed to a more easily readable and interpretable form. Third, it does not allow for patient altering or doctoring of records. Fourth, it is encrypted and username protected. Fifth, it is HIPAA compliant. Sixth, it allows quick and easy access to medical records in emergent situations.

### BRIEF DESCRIPTION OF THE DRAWINGS

The objects and features of the present invention will be best understood by a detailed description of a preferred embodiment thereof, selected for the purposes of illustration and shown in the accompanying drawings:

FIG. 1 shows an overview of the flow of data, processing of the medical data files, and output of the reviewed medical records to a portable media device or through display via the network;

FIG. 2 shows the flow of original medical records to be reviewed by a physician and storage on the medical database;

FIG. 3 shows the flow of the reviewed medical data file to a remote computer for the requesting physician;

FIG. 4 shows the flow of the reviewed medical record file to the portable digital memory device;

FIG. 5 shows the flow of a electronic medical record to a review by a physician and storage on the medical database;

FIG. 6 shows the physician portal for viewing the original medical records and summarizing the medical record in data fields; and

FIG. 7 shows the medical chart as viewed through a remote computer or on the portable digital memory device.

### DETAILED DESCRIPTION OF PREFERRED EMBODIMENTS

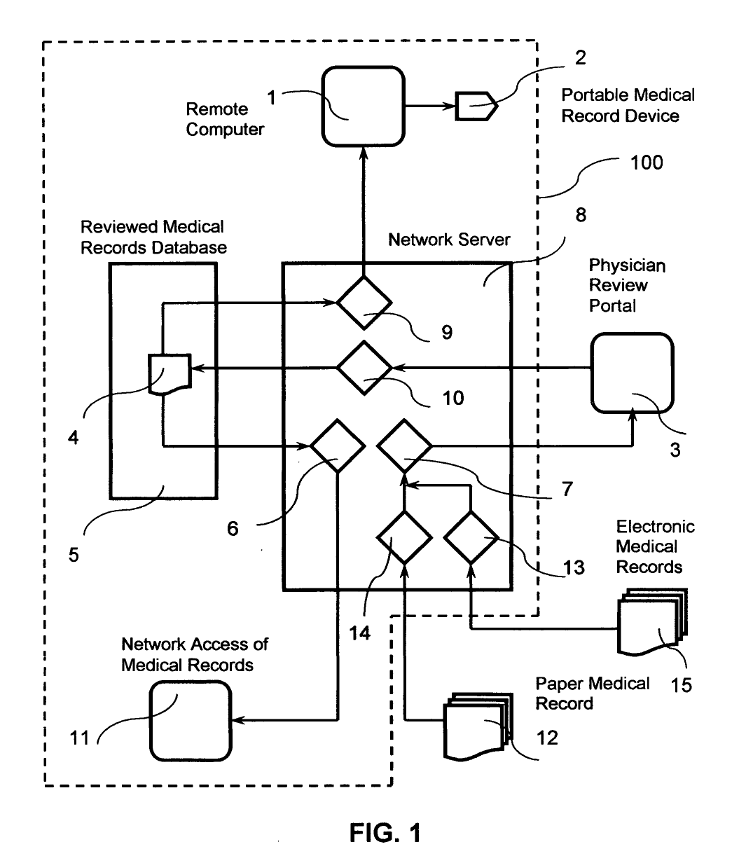

Referring first to FIG. 1, an overview of the health information management and review system of the present invention is shown. The system comprises of paper medical records 12, electronic medical records 15, a network server 8, several remote computers 1, 3, 11 connected to the network server 8, a medical record database 5, and software programs 6, 7, 9, 10, 13, 14 executing from the network server 8, and a portable medical record device 2. A network 100 consisting of remote computers 1, 3, 11, a network server 8 with software programs 6, 7, 9, 10, 13, 14 executing from network server 8, medical record database 5 can be the internet or intranet. The flow of data is shown schematically and will be discussed in detail in the following figures.

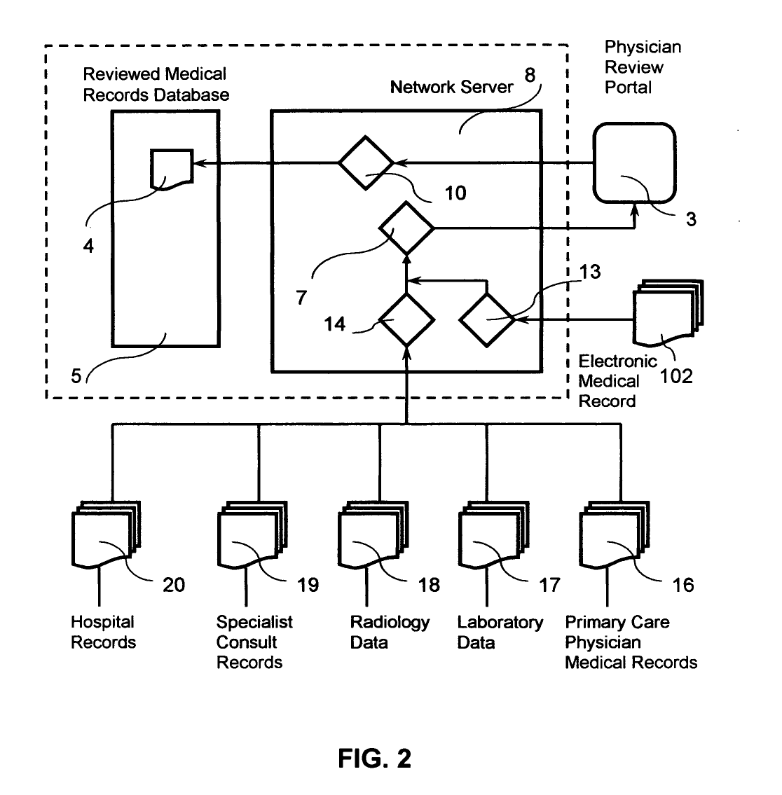

Referring to FIG. 2, the method of receiving medical records 16-20, the review of medical records 16-20, and storing the reviewed medical records onto medical record database 5 is detailed. Prior to records being sent, a person signs a release of records to have all of their medical records 16-20 sent to network server 8 through fax or electronic transfer. Paper medical records 16-20 from different sources and different types can be faxed to network server 8. Examples of medical records 16-20 can include, but are not limited to, primary care physician medical records 16, laboratory data 17, radiology data 18, specialist consult records 19, or hospital records 20. Paper records are defined in this setting as any record that can be printed on paper or transferred to paper or displayed in a digital form that is not part of an electronic medical record. Paper medical records 16-20 are faxed or e-mailed and recorded in a digital format on network server 8 through software program 14. Software program 14 sends the record to software program 7 that coordinates the transfer to remote computer 3 for physician review. Software program 7 also displays medical record 16-20 for the physician to review. The physician enters the summarized medical record 4 information into the remote computer 3, the data is sent to software program 10 which receives the file and stores the reviewed medical record 4 on the reviewed medical record database 5. Electronic medical records 102 are received by software program 13. Software program 13 extracts the core elements of the medical record 102 and saves the file temporarily. The record is then sent to software program 7 which again coordinates the transfer to and displays the record on remote computer 3 for physician review. After physician review, the data is sent to software program 10 which receives the file 102 and stores reviewed medical record 4 on the reviewed medical record database 5. Software program 10 also encrypts reviewed medical record 4.

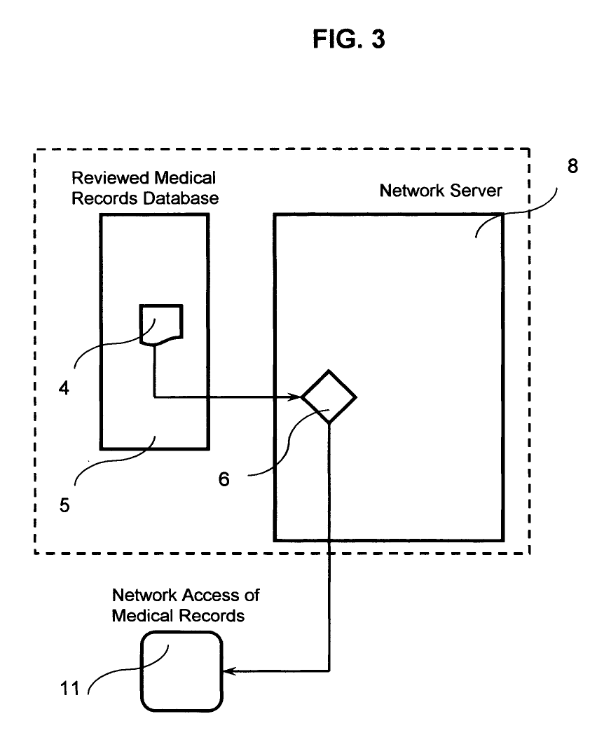

Referring to FIG. 3, the method of transferring and displaying the medical records 4 to a requesting physician is outlined. In an emergency, a person who subscribes to the service can have their medical records 4 displayed and transferred to remote computer 11 through network 100, preferably the internet. A worn piece of jewelry such as a bracelet or a necklace with instructions to access medical records 4 through a website address or phone number. The treating physician calls the phone number and an operator verifies the requesting physicians information (DEA number, state medical license number, hospital, phone, address, e-mail, reason for the request) along with the medical record number located on the piece of jewelry of the patient. The physician is then instructed to go to the website and enter a username and password. Also, the physician can go to the website directly and enter the physician's personal information and medical record number of the patient. Once the username and password is entered, the physician has full access to all of the patient's medical record 4 for short period of time, for example, 24 hours. After the elapsed time, the username and password will change. The reviewed medical record 4 on reviewed medical record database 5 is sent through software program 6 to be displayed on remote computer 11 for physician to review.

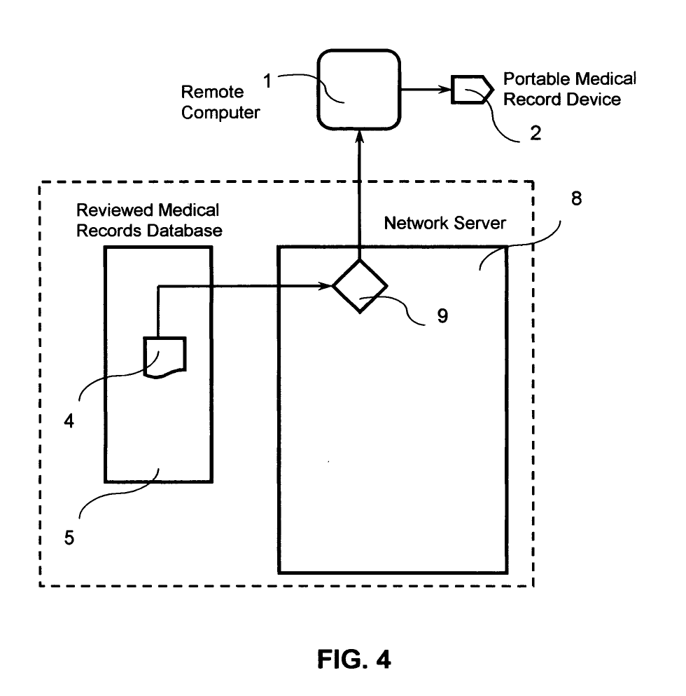

Referring to FIG. 4, the method of transfer of reviewed medical record 4 to portable digital memory device 2 is outlined. A person, after visiting their physician, can contact the service through e-mail, a phone number, or going to a website and request medical records 4 be updated. The person gives the name and information of the physician who they saw or is recalled from stored information they entered when they first subscribe to the service. A request for medical records 16-20, 102 of FIG.2 is sent directly to the physician's office, hospital, laboratory. Medical records 16-20, 102 are transferred as according to FIG. 2, as detailed in the previously. During the initial review, portable digital memory device 2 is sent directly to the patient. Subsequently, the updates after can be transferred through network 100. A person after requesting an update receives notification that their update is ready to download via a phone call, e-mail, text message on their cellular phone. The patient then logs on to a website and inserts portable digital memory device 2 into remote computer 1. The person then hits a transfer button on the website and reviewed medical record 4 is transferred from reviewed medical records database 5 onto remote computer 1 via software program 9 directly onto portable digital memory device 2.

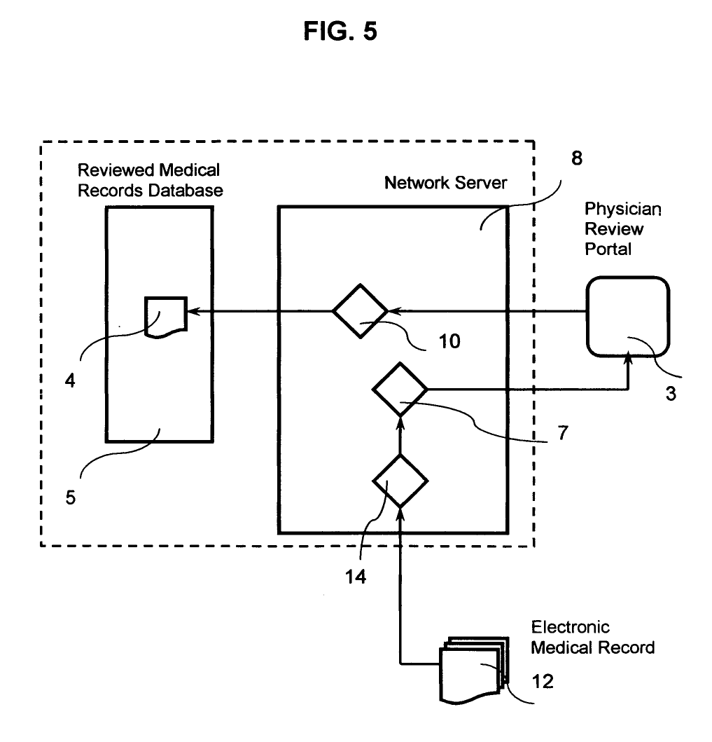

Referring to FIG. 5, the method of extracting core elements of medical record 4 is outlined. With electronic medical records 12, a complete medical record is already available in electronic format. This may be too cumbersome to carry on portable medical record device 2. As electronic medical records formats become standard as mandated by the government, it will be easier to create a one or a few software programs that extracts core elements found in a complete medical record to form a summarized version of medical record 4 for ease and speed of use by physicians. Electronic medical record 12 is sent to network server 8 to receiving and processing program 14. Software program 14 temporarily stores the information and then extracts key data fields from electronic medical record 12 and transfer the information to software program 7 to be displayed on remote computer 3 for physician review. The reviewing physician can alter or modify record 12 to clarify or correct mistakes made by the extraction program 14. The reviewed medical record from electronic medical record 12 is then sent to software program 10 and stored and cataloged as a reviewed medical record 4 in the reviewed medical records database 5.

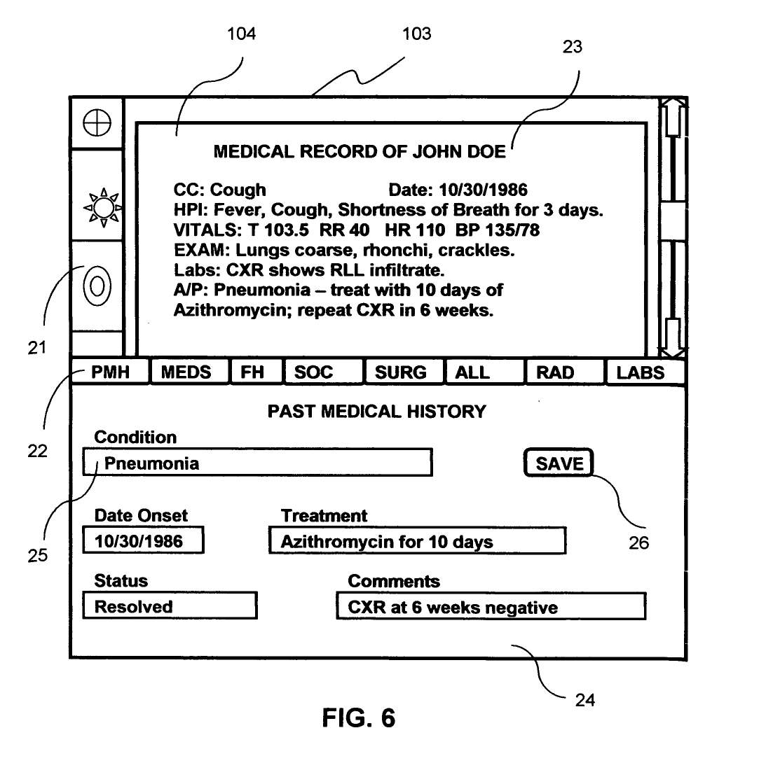

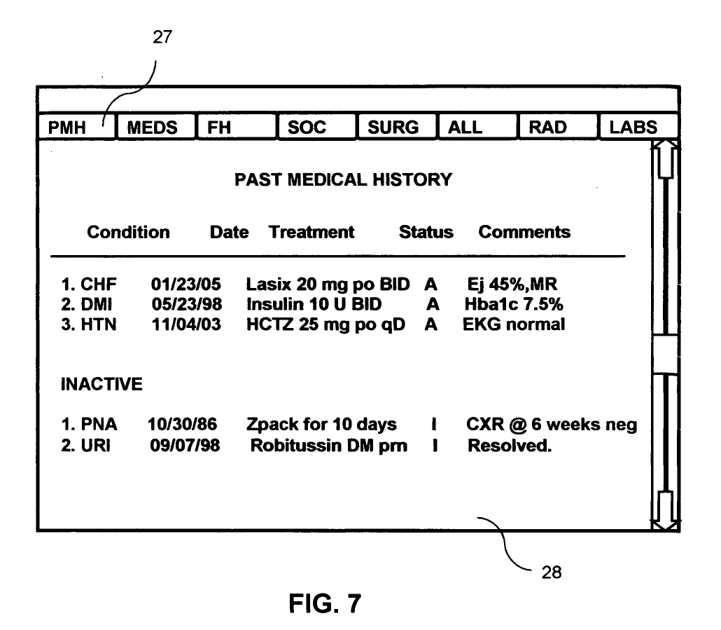

Referring to FIG. 6, a physician review portal 24 is shown. The original medical record 23 is displayed in the top portion of the screen 104. These records can be recorded in digital format such as TIFF, JIFF, PDF and displayed on screen 104 of physician review portal 24. On the bottom half, the reviewing physician enters the summarized medical information after reviewing the original medical records 23 from the top portion. Tabs on the top of the data entry screen 22 allows for easy navigation of sections of reviewed medical record 28 of FIG. 7. After the physician has completed the element of data entry, they can hit save button 26. The information is saved on medical record database 5. There are also tools to help view the original medical record 21. In this example, medical record 23 from a physician's office for “John Doe” is displayed. It shows he has a pneumonia. Only key pieces of information is retained in reviewed medical record 28 of FIG. 7. In this case, for past medical history, the condition, the date of onset, the treatment, status outcome, and comments are pertinent in the reviewed medical record 28. The tabs on the top of the data entry screen 22 are only a few examples for illustration.

Referring to FIG. 7, an example of the reviewed medical record 28 is shown. Each of the different headings of the reviewed medical record are tabs 27 at the top. By selecting each of tabs 27, the information from the heading selected is displayed. In this example, past medical history 27 is selected and different medical conditions are displayed. They include information such as the name of the disease or condition, date of onset, the treatment, status of outgoing disorder, and key comments pertaining to the illness. Also, inactive medical problems are shown. These are all illustrative examples.

The present invention has been described in terms of specific embodiments. These embodiments have been provided for illustrative purposes and are not to be used to limit the scope of the present invention. As those skilled in the art will understand, one or more features of the above embodiments may be changed, modified, or altered without departing from the scope of the present invention.

I claim:

1. A system of processing and managing personal health information comprising:
a portable digital memory device;
a network server including a secured database of medical records; and
a physician portal application.

2. The system of claim 1 wherein the management of the personal health information is executed through software on the server and the portable digital memory device.

3. The system of claim 1 wherein the physician portal application allows for review and summary of medical records by physicians.

4. The system of claim 1 wherein the digital memory device can be carried by a person on a selected one of a key chain, in a wallet and as a piece of jewelry.

5. The system of claim 1, wherein visual indicia on bracelet or a necklace alerts emergency personnel with instructions to obtain information to access to the personal health information through a phone number or through an internet website.

6. The system of claim 1, wherein the network database includes internet and an intranet.

7. The system of claim 1 wherein said portable digital memory device comprising: a processor and a digital memory linked with the processor.

8. The system of claim 7 wherein said digital memory comprising a viewer application software program.

9. The system of claim 2 wherein the software program comprising:
means for providing for reading the said medical data file and displaying the contents on a monitor;
means for providing execution without installing software on a remote computer;
means for providing protection of personal medical information through a username and password; and
means of preventing alteration or doctoring of medical records by the patient by allowing a “read only” feature of the said medical data file.

10. The system of claim 2, wherein a medical & personal information data stored in an encrypted format, a medical & personal information data stored in text and imaging formats and an external connector that interfaces with a computer.

11. The system of claim 2 wherein said external connector has an interface for easily detachable connection with a computer such as USB port and a rigid housing unit adapted to house said processor, said digital memory, and said external connector to a computer.

12. The system of claim 1 the network database and server comprising:
a managing software application with means to manage and catalog medical data files;
a downloading software application with means to download updated medical data file to a remote computer and onto the portable digital medical record device;
a remote computer viewing software application with means to allow viewing the contents of the medical data file on a remote computer for a requesting physician;
a receiving data software application with means to store the sent medical records, imaging, laboratory reports, letters, pathology reports and que the files to the appropriate physician to review;
a physician review software application with the means to display the medical record files through a secured connection to a remote computer for a reviewing physician to view and alloy a the reviewing physician to input the summarized information into a portal that allows transfer of saved data to said network database;
an extraction software application to pull data fields from an electronic medical record and send the information to a viewer application; and
a network database containing the medical data profile of people.

13. The system of claim 1 wherein said medical information is encrypted.

14. The system of claim 1 wherein the secured connection between said reviewing physician and said network database can be secured through VPN.

15. The system of claim 1 comprising:
a server based viewer application operating on a remote computer to view medical records;
a physician data entry portal to enter reviewed and summarized health data into the network database; and
a method to allow easy transfer of images and reports directly to the medical data file.

16. The system of claim 1 wherein an electronic health record extraction application comprising: a software application that extracts core medical information from an electronic medical record and stores in the said internet database.

17. A system for managing, storing and viewing medical records comprising:
a network server having a medical record database, the network server being connected to a network; and
a portable digital memory device connects to the network, the portable digital memory device has software for accessing the medical records database via the network server upon entry of an identification and a password.

18. The system of claim 1 wherein the management of the personal health information is executed through software on the server and the portable digital memory device and the physician portal application allows for review and summary of medical records by physicians.

19. The system of claim 17 wherein the network database and server comprising:
a managing software application with means to manage and catalog medical data files;
a downloading software application with means to download updated medical data file to a remote computer and onto the portable digital medical record device;
a remote computer viewing software application with means to allow viewing the contents of the medical data file on a remote computer for a requesting physician;
a receiving data software application with means to store the sent medical records, imaging, laboratory reports, letters, pathology reports and que the files to the appropriate physician to review;
a physician review software application with the means to display the medical record files through a secured connection to a remote computer for a reviewing physician to view and alloy a the reviewing physician to input the summarized information into a portal that allows transfer of saved data to said network database;
an extraction software application to pull data fields from an electronic medical record and send the information to a viewer application; and
a network database containing the medical data profile of people.

20. A method for processing and managing personal health information comprising:
storing patient information on a network server including a secured database of medical records,
reviewing the patient information by a physician using a physician portal application to make a summary; and
retrieving the patient information and summary with the portable digital memory device by a user the user is a heath care professional.
